(19) United States
(12) Patent Application Publication
(10) Pub. No.: US 2006/0029698 A1
(43) Pub. Date: Feb. 9, 2006

(54) Food template

(76) Inventors: Trent Watson, Stockton (AU); Rhys Collins, Valentine (AU)

Correspondence Address: Jay F. Moldovanyi, Esq.;Fay, Sharpe, Fagan, Minnich & McKee, LLP, Seventh Floor, 1100 Superior Avenue, Cleveland, OH, 44114-2579, US

(21) Appl. No.: 11/190,044

(22) Filed: Jul. 26, 2005

(30) Foreign Application Priority Data
Aug. 3, 2004 (AU) 2004100628

(51) Int. Cl. G01N 33/02

(52) U.S. Cl. 426/231

(57) ABSTRACT

A food template (10) is adapted to be removably applied to a food bearing surface of a food holder, such as a plate (20). The food template (10) physically defines a plurality of areas, each signifying the allowable quantity of food to be placed in the respective area. In one embodiment, the template (10) comprises an outer raised ring (11) and a plurality of raised radial arms (12) within the ring. Each of the food areas is defined between the ring and an adjacent pair of the arms. The template (10) includes indicia thereon indicating the type of food for each area.

This invention relates to a food portioning template. In particular, the invention relates to a template adapted for use on a food holder, such as a plate, for use in managing a person's diet. The food portioning template facilitates practical eating solutions for an individual.

### BACKGROUND ART

Although most persons recognise the benefits of eating the correct quantities and types of foods, they often fail to implement the necessary practical measures to achieve the benefits of healthy eating. For example, persons may fail to appreciate or understand which types of foods are suitable for their age, size, weight, sex or other personal characteristics. Persons may also find it difficult to judge the appropriate quantity of food portions which they should eat.

It is well known to use diet plans to manage a person's food intake. However, unless the diet plan is immediately on hand, a person may forget which types and/or quantities of foods he or she should be eating.

It is also known to use a “diet plate” for portion control (www.thedietplate.com). The diet plate is a plate with line markings thereon designed to indicate the portions of different types of foods to be eaten. The markings define sections, including a carbohydrate section, a protein section, and a sauce circle. The diet plate is calibrated so that a particular section can hold no more than a prescribed quantity before the food starts to slide over the boundary of that section. The diet plate is intended to replace the normal dinner plate, and the markings are applied permanently to the diet plate so that it is dishwasher safe.

Although the diet plate is useful for portioning food groups on a dinner plate, it has several disadvantages. Its principal disadvantage is that it is not transferable to ordinary plates. Hence, the person must always ensure that he or she has the diet plate at meal time. This may be inconvenient where the person is away from home. Secondly, the diet plate concept is not transferable to a person's existing plates within the home. Thirdly, the application of permanent markings to an earthenware plate to make it both microwave and dishwasher safe makes the diet plate relatively expensive. Finally, the diet plate does not always apportion food accurately as the line markings on the diet plate do not restrain the food and they can be covered.

It is an aim of the present invention to provide a food template which overcomes or substantially ameliorates one or more of the above described disadvantages, or which at least provides a useful choice.

### SUMMARY OF THE INVENTION

In one broad form, the present invention provides a food template adapted to be removably applied to a food bearing surface of a food holder, such as a plate. The food template comprises a raised peripheral portion defining a boundary, and at least one raised divider portion within the peripheral portion and joined thereto, the divider portion(s) physically defining a plurality of separate areas within the boundary, each area having an open top and an open bottom. The size of each area signifies the maximum quantity of a food or food type to be placed in that area.

Typically, the peripheral portion is an outer ring, and the divider portion(s) comprise a plurality of radial arms, each of the areas being defined between the outer ring and an adjacent pair of the radial arms. The peripheral portion may be of angle section.

A hub portion may be provided at the centre of the template, with each radial arm extending from the hub portion to the outer ring.

Preferably, the template has indicia formed on the peripheral portion indicating food types for each respective area.

Advantageously, the radial arms have angled underside edges.

In another form, the invention provides a method of portioning food on a plate, comprising placing a template as described above on the plate, and placing portions of food types in the areas, the allowable size of each portion being determined by the size of its respective area. The type of food placed in an area is governed by the indicia formed on the template.

In order that the invention may be more fully understood and put into practice, a preferred embodiment thereof will now be described with reference to the accompanying drawings.

### BRIEF DESCRIPTION OF THE DRAWINGS

FIG. 1 is a top perspective view of a template according to one embodiment of the invention.

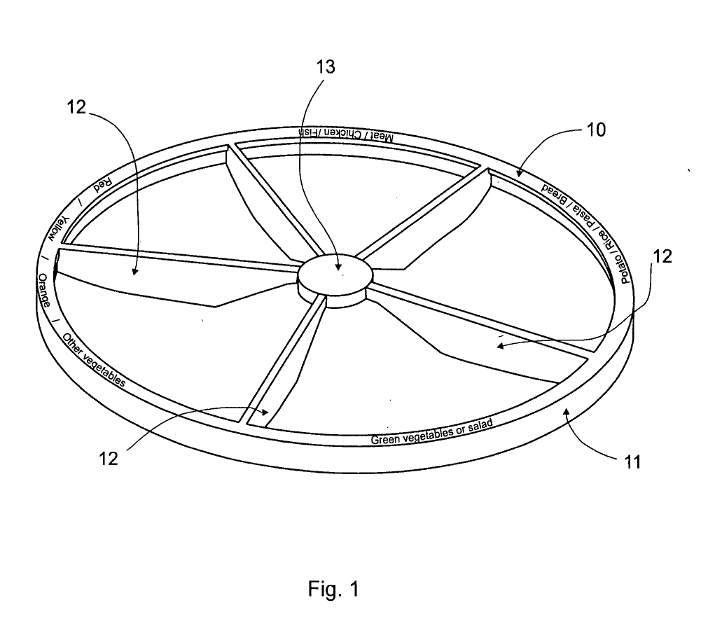

FIG. 2 is a bottom perspective view of the template of FIG. 1.

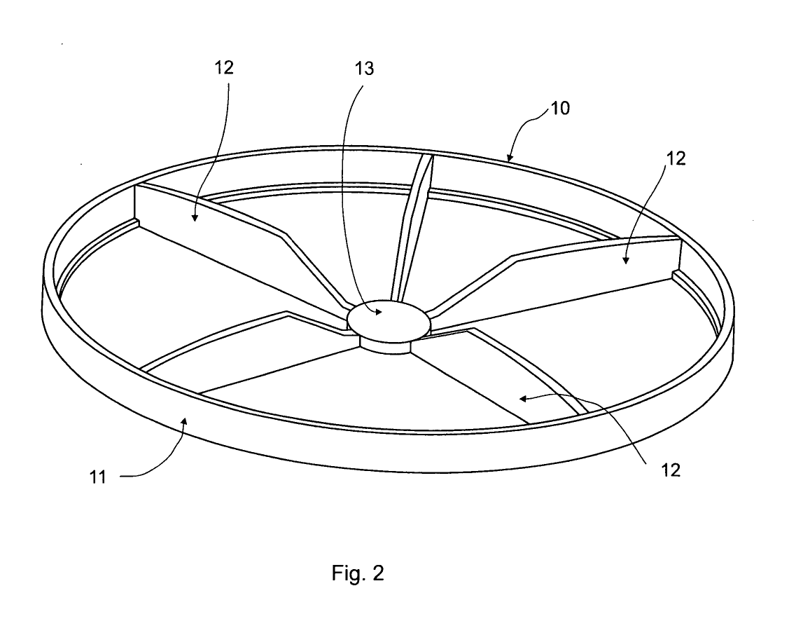

FIG. 3 is a plan view of the template of FIG. 1 in use, applied to a plate.

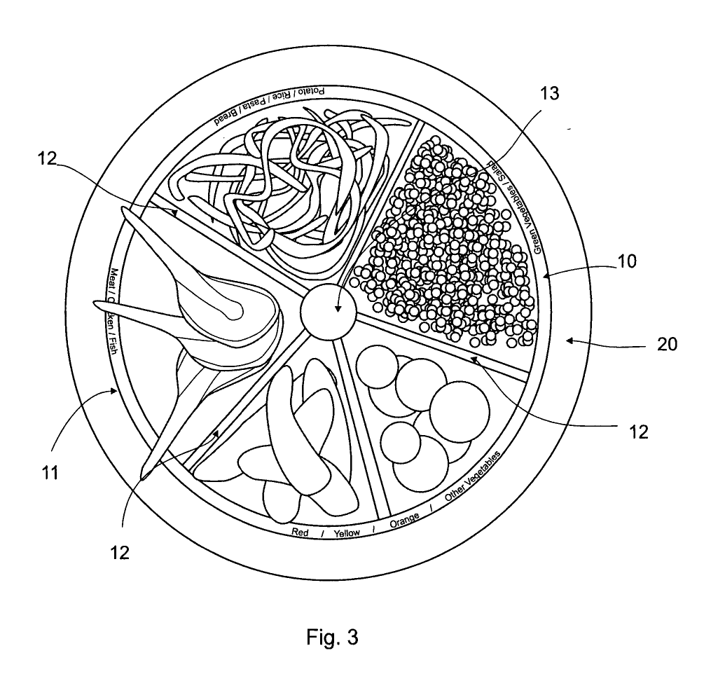

### DESCRIPTION OF PREFERRED EMBODIMENTS

As shown in the drawings, a food template 10 comprises an outer ring 11 and a plurality of radial arms 12 which extend from an inner hub 13 to the outer ring 11. The radial arms 12 divide the circle defined by the outer ring 11 into a plurality of segments or food areas, each of which has an open top and an open bottom.

The outer ring 11 is raised. It is of angle section and forms a low outer wall. The radial arms 12 are also raised. They are formed by upright strips which form low dividing walls.

A hub portion 13 is provided at the centre of the ring 11. The radial arms 12 extend outwardly from the hub portion 13 to the ring 11.

The template 10 is adapted to be placed on a plate 20. The underside edges of the radial arms 12 are angled to enable the template 10 to sit or rest stably on dinner plates of different sizes and shapes.

In use, the area or space between a pair of adjacent arms 12 and the outer ring 11 defines the size or quantity of a respective type of food to be placed on the plate. The permissible type of food is indicated by indicia formed on the outer ring 11 adjacent the respective area. The template 10 therefore provides a reliable guide for the correct portioning of food types, such as meats, carbohydrates and vegetables or salad.

The template 10 may suitably be made of plastic by injection moulding, or formed of stainless steel or other suitable material.

The template 10 may be made in different sizes, with different area sizes, to suit different users, e.g. children, adolescents, athletes, middle aged persons and elderly persons.

The template 10 may be used in conjunction with a measuring cup to provide portion control for all daily meals. The measuring cup (not shown) can be used to apportion breakfast foods, such as by measuring the quantity of cereal, milk and beverages to be eaten at breakfast, while the template 10 provides an indication of the type and quantity of foods for lunch and dinner.

The above described template has several advantages, including
It assists the user in making correct choices in relation to the type and quantity of foods, thereby providing a healthy eating regime.
The template is simple and economical to manufacture.
The template is low cost.
The template is simple to use.
The template provides a practical visual guide to nutrition.
The template is portable and reusable.

The foregoing describes only one embodiment of the invention, and modifications which are obvious to those skilled in the art may be made thereto without departing from the scope of the invention as defined in the following claims.

For example, the template may be constructed as a single use, disposable, device.

Furthermore, the outer ring need not be circular, and the arms need not be radial.

1. A food template adapted to be removably applied to a food bearing surface of a food holder, such as a plate, the food template comprising
a raised peripheral portion defining a boundary, and
at least one raised divider portion within the peripheral portion and joined thereto, the divider portion physically defining a plurality of separate areas within the boundary, each area having an open top and an open bottom,
wherein the size of each area signifies the maximum quantity of a food or food type to be placed in that area.

2. A food template as claimed in claim 1, wherein the peripheral portion is an outer ring, and the divider portion comprises a plurality of radial arms, each of the areas being defined between the outer ring and an adjacent pair of the radial arms.

3. A food template as claimed in claim 2, further comprising a hub portion at the centre of the template, each said radial arm extending from the hub portion to the outer ring.

4. A food template as claimed in claim 3, wherein the peripheral portion, divider portion(s) and hub portion are integrally formed.

5. A food template as claimed in claim 2, wherein the radial arms have angled underside edges.

6. A food template as claimed in claim 1, wherein the template has indicia formed on the peripheral portion indicating food types for each respective area.

7. A food template as claimed in claim 6, wherein the peripheral portion is of angle section.

8. A food template adapted to be removably applied to a plate, the template comprising a peripheral ring member no larger than the plate, and a plurality of radial arms dividing the space within the ring member into a plurality of areas, each area being defined between the outer ring and an adjacent pair of the radial arms and having an open top and an open bottom, and wherein the ring member and the radial arms form low walls at least partially around each area, and the size of each area determines the maximum quantity of a food to be placed in that area.

9. A food template as claimed in claim 8, further comprising a hub portion at the centre of the template, each said radial arm extending from the hub portion to the outer ring.

10. A food template as claimed in claim 8, wherein the template has indicia formed on the peripheral portion indicating at least one food type for each respective area.

11. A method of portioning food on a plate, comprising
placing a template on the plate, the template comprising
a peripheral ring member no larger than the plate, and a plurality of radial arms dividing the space within the ring member into a plurality of areas, each area being defined between the outer ring and an adjacent pair of the radial arms and having an open top and an open bottom, and wherein the ring member and the radial arms form low walls at least partially around each area; and
placing portions of food types in the respective areas, the allowable size of each portion being determined by the size of its respective area.

12. A method as claimed in claim 11, wherein the type of food placed in an area accords with indicia formed on the peripheral portion indicating permissible food types for each respective area.
